(19) United States
(12) Patent Application Publication
(10) Pub. No.: US 2005/0189743 A1
(43) Pub. Date: Sep. 1, 2005

(54) Inflatable curtain with extension portion

(75) Inventors: Ali Emam Bakhsh, Rochester Hills, MI (US); Ayad G. Nayef, Sterling Heights, MI (US); Navin Chopra, Rochester Hills, MI (US)

Correspondence Address: TAROLLI, SUNDHEIM, COVELL & TUMMINO L.L.P.;1111 LEADER BLDG., 526 SUPERIOR AVENUE, CLEVELAND, OH, 44114-1400, US

(73) Assignee: TRW Vehicle Safety Systems Inc.

(21) Appl. No.: 10/787,985

(22) Filed: Feb. 26, 2004

(51) Int. Cl. A61M 29/00

(52) U.S. Cl. 280/730.2; 280/743.1

(57) ABSTRACT

An inflatable vehicle occupant protection device (14) is inflatable to a position between a side structure (16) of a vehicle (12) and a vehicle occupant. The protection device (14) includes an inflatable main portion (50) inflatable to cover a portion of the vehicle side structure (16) including a front side window (40) and a rear side window (42). The main portion (50) has an upper edge (70) and a lower edge (72) extending along the length of the main portion and front and rear portions (74 and 76) spaced apart along the length of the main portion. The main portion (50) comprises overlying panels (52) that are interconnected to form a seam (58) extending along the upper and lower edges (70 and 72) and along the front and rear portions (74 and 76). The inflatable curtain (14) also includes an extension portion (100) comprising a separate panel (102) interconnected with the main portion (50) along the seam (58). The extension portion (100) covers an additional portion of the vehicle side structure (16).

### FIELD OF THE INVENTION

The present invention relates to an inflatable apparatus for helping to protect a vehicle occupant in the event of a side impact to the vehicle and/or a vehicle rollover.

### BACKGROUND OF THE INVENTION

It is known to inflate an inflatable vehicle occupant protection device to help protect a vehicle occupant upon the occurrence of an event for which occupant protection is desired. One particular type of inflatable vehicle occupant protection device is an inflatable curtain. The inflatable curtain is inflatable away from the roof of the vehicle between a vehicle occupant and the side structure of the vehicle in response to a side impact to the vehicle and/or a vehicle rollover. A known inflatable curtain is inflated from a deflated condition with inflation fluid directed from an inflator to the inflatable curtain.

### SUMMARY OF THE INVENTION

The present invention relates to an inflatable vehicle occupant protection device is inflatable to a position between a side structure of a vehicle and a vehicle occupant. The protection device includes an inflatable main portion inflatable to cover a portion of the vehicle side structure including a front side window and a rear side window. The main portion has an upper edge and a lower edge extending along the length of the main portion and front and rear portions spaced apart along the length of the main portion. The main portion comprises overlying panels that are interconnected to form a seam extending along the upper and lower edges and along the front and rear portions. The inflatable curtain also includes an extension portion comprising a separate panel interconnected with the main portion along the seam. The extension portion covers an additional portion of the vehicle side structure.

The present invention also relates to an inflatable vehicle occupant protection device inflatable away from a roof of a vehicle to a position between a side structure of the vehicle and a vehicle occupant. The inflatable vehicle occupant protection device includes an inflatable main portion inflatable to cover a portion of the vehicle side structure including a front side window of the vehicle and a rear side window of the vehicle. The main portion has upper and lower edges extending along its length and front and rear edges extending between the upper and lower edges. The main portion is woven as a single piece of material and includes a woven seam extending along said edges. The inflatable vehicle occupant protection device also includes an extension portion woven as a single piece of material separate from the main portion. The extension portion is interconnected with the main portion along the seam and covers an additional portion of the vehicle side structure.

The present invention also relates to an apparatus for helping to protect an occupant of a vehicle that has a side structure including front and rear side windows and a roof. The apparatus includes an inflatable curtain that is inflatable away from the vehicle roof to a position adjacent the side structure of the vehicle. The inflatable curtain includes an inflatable main portion inflatable to cover a portion of the vehicle side structure between the front side window and a front vehicle occupant and between the rear side window and a rear vehicle occupant. The main portion has upper and lower edges extending along its length and front and rear edges extending between the upper and lower edges. The main portion includes overlying panels that are interconnected to form a seam extending along the edges. An extension portion includes a separate panel of material interconnected with the main portion along the seam and extending along at least one of the edges. The extension portion covers an additional portion of the vehicle side structure.

The present invention also relates to an apparatus including an inflatable vehicle occupant protection device inflatable away from a roof of a vehicle to a position between a side structure of the vehicle and a vehicle occupant. The inflatable vehicle occupant protection device includes a lower edge extending along its length. A lower extension portion extends from the lower edge along a portion of the length of the inflatable vehicle occupant protection device. The lower extension portion is adapted to cover a portion of the side structure where a thorax of a vehicle occupant may be positioned.

### BRIEF DESCRIPTION OF THE DRAWINGS

The foregoing and other features of the present invention will become apparent to one skilled in the art to which the present invention relates upon consideration of the following description of the invention with reference to the accompanying drawings, in which:

FIG. 1 is a schematic view of an apparatus for helping to protect a vehicle occupant illustrating the apparatus in a deflated condition in a vehicle, according to a first embodiment of the present invention;

FIG. 2 is a schematic view of the apparatus of FIG. 1 in an inflated condition in the vehicle;

FIG. 3 is a sectional view taken generally along line 3-3 in FIG. 2;

FIG. 3A is a sectional view similar to FIG. 3 illustrating an alternative construction of the apparatus;

FIG. 4 is a plan view illustrating a portion of the apparatus of FIGS. 1-3A in a disassembled condition; and

FIGS. 5-10 are schematic views of apparatuses for helping to protect a vehicle occupant, according to different embodiments of the present invention.

### DESCRIPTION OF EMBODIMENTS

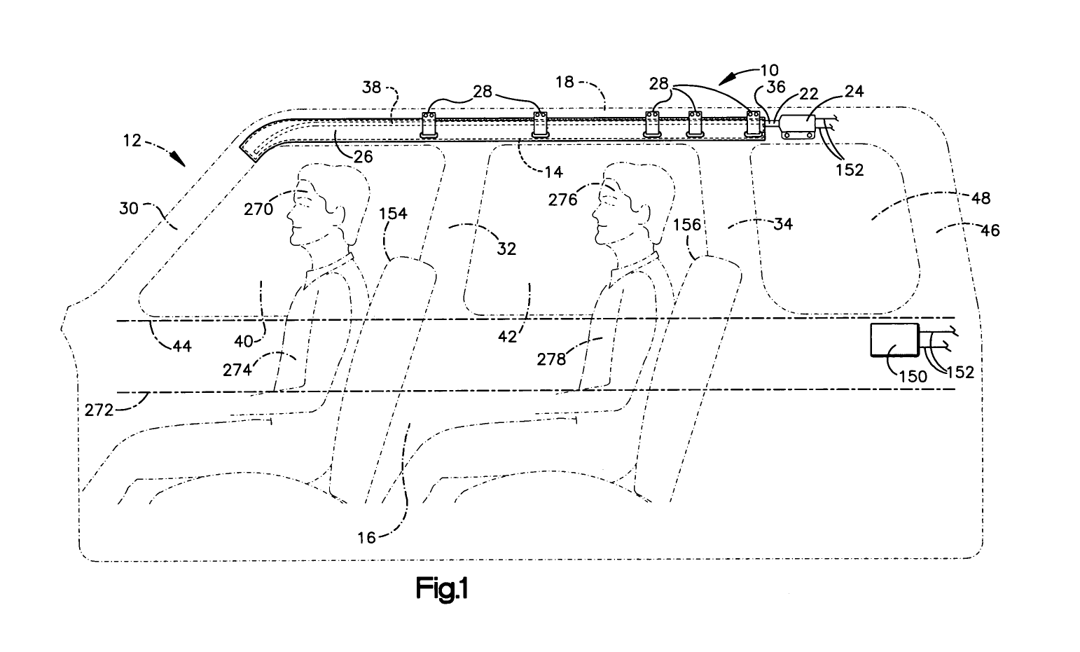

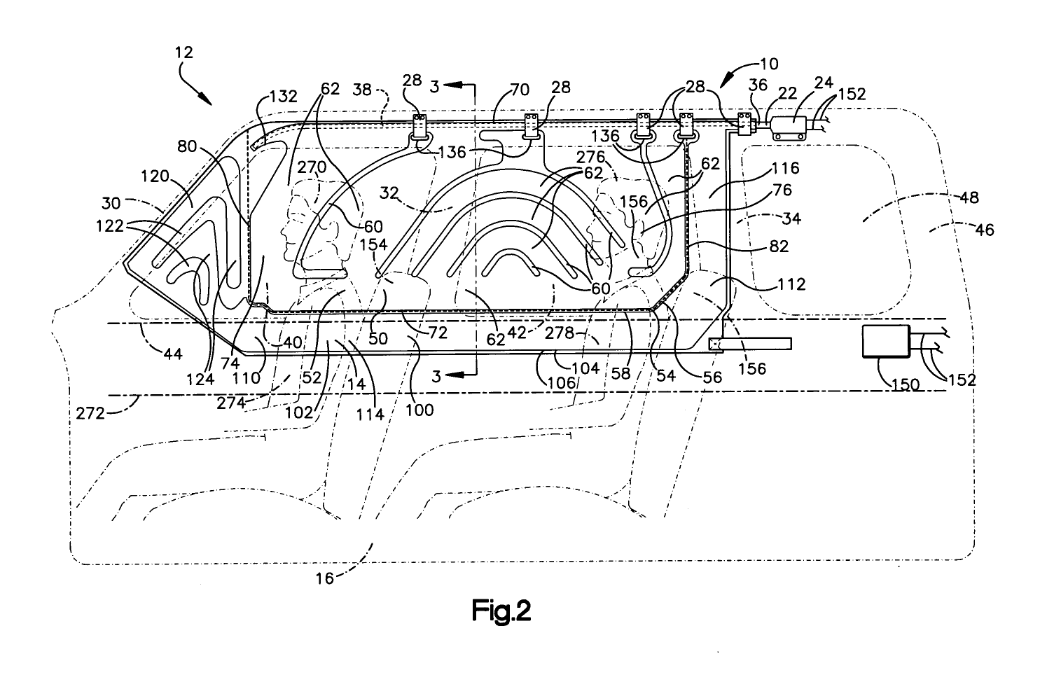

Representative of the present invention, an apparatus 10 helps to protect an occupant 270, 276 of a vehicle 12. As shown in FIGS. 1 and 2, the apparatus 10 includes an inflatable vehicle occupant protection device in the form of an inflatable curtain 14 that is mounted adjacent the side structure 16 of the vehicle 12 and the roof 18 of the vehicle. The side structure 16 of the vehicle 12 includes front and rear side windows 40 and 42, an A pillar 30, a B pillar 32, and a C pillar 34. The side structure 16 may also include a D pillar 46 and a side window 48 positioned rearward of the side windows 40 and 42. Referring to FIGS. 1-3, the vehicle 12 also includes a belt line 44, which is defined by an imaginary line that extends along the length of the vehicle in the general location of the lower edge or sill of the side windows 40, 42, and 48.

An inflator 24 is connected in fluid communication with the inflatable curtain 14 through a fill tube 22. The fill tube 22 may be constructed of any suitable material, such as metal, plastic, or fabric. Also, those skilled in the art will appreciate that the fill tube 22 may be omitted, in which case the inflator 24 may be connected directly to the inflatable curtain 14. The fill tube 22 has a first end portion 36 for receiving fluid from the inflator 24. The fill tube 22 may be connected directly to the inflator 24 or a manifold (not shown) may connect the fill tube to the inflator. The fill tube 22 has a second end portion 38 disposed in the inflatable curtain 14.

The inflator 24 contains a stored quantity of pressurized inflation fluid (not shown) in the form of a gas to inflate the inflatable curtain 14. The inflator 24 alternatively could contain a combination of pressurized inflation fluid and ignitable material for heating the inflation fluid, or could be a pyrotechnic inflator that uses the combustion of gas-generating material to generate inflation fluid. As a further alternative, the inflator 24 could be of any suitable type or construction for supplying a medium for inflating the inflatable curtain 14.

The apparatus 10 may include a housing 26 (FIG. 1) that stores the inflatable curtain 14 in a stored and deflated condition. The fill tube 22, the deflated inflatable curtain 14, and the housing 26 have an elongated configuration and extend along the vehicle roof 18 and along the side structure 16 of the vehicle 12 above the side windows 40, 42, and 48. The fill tube 22, inflatable curtain 14 and housing 26 are connected to the vehicle 12 by known connectors 28, such as brackets.

Referring to FIGS. 2-4, the inflatable curtain 14 includes a main portion 50 and an extension portion 100. The main portion 50 includes panels 52 of material that are arranged in an overlying manner. Overlapping portions of the panels 52 are secured together by weaving the panels together along at least a portion of a perimeter 54 of the main portion 50 to form a perimeter connection 56 of the main portion. The perimeter connection 56 helps define an inflatable volume of the main portion 50. The perimeter connection 56 also helps define a seam 58 that extends along the perimeter 54 of the main portion 50.

The main portion 50 of the inflatable curtain 14 may also include interior connections 60 in which the overlying panels 52 are interconnected within the perimeter 54 of the main portion. The interior connections 60 form non-inflatable portions of the main portion 50 within the perimeter 54 of the main portion. The interior connections 60 also help define inflatable chambers 62 of the main portion 50. The configuration of the interior connections 60, and thus the chambers 62, may vary depending on a variety of factors, such as the architecture of the vehicle 12, the position of the inflatable curtain 14 and/or main portion 50 in the vehicle, and the desired extent or coverage of the inflatable curtain and/or main portion.

The panels 52 may be woven from a material, such as nylon yarn, and may be coated with a gas impermeable material, such as urethane, or laminated with a gas impermeable film. The main portion 50 of the inflatable curtain 14 thus may have a substantially gas-tight construction. Those skilled in the art will appreciate that alternative materials, such as polyester yarn, and alternatives coatings, such as silicone, may also be used to construct the main portion 50.

The perimeter 54 of the main portion 50 is defined at least partially by an upper edge 70, an opposite lower edge 72 of the curtain, and front and rear portions 74 and 76, respectively, of the main portion spaced apart horizontally along the upper and lower edges. The front and rear portions 74 and 76 of the main portion include front and rear edges 80 and 82, respectively, that are spaced horizontally apart along the upper and lower edges 70 and 72 and extend between the upper and lower edges. Those skilled in the art will appreciate that the main portion 50 may have a shape that differs from that of the inflatable curtain 14 of the illustrated embodiment.

In the embodiment illustrated in FIGS. 2-4, the extension portion 100 includes panels 102 of material that are arranged in an overlying manner. Overlapping portions of the panels 102 are secured together by, for example, weaving the panels together along at least a portion of a perimeter 104 of the extension portion 100 to form a perimeter connection 106 of the extension portion. Alternatively, the panels 102 could be separate pieces of material sewn together along their perimeters. The perimeter connection 106 helps define an inflatable volume of the extension portion 100. The perimeter connection 106 also helps define a seam 108 of the extension portion 100.

Like the panels 52 of the main portion 50, the panels 102 of the extension portion 100 may be woven from a material, such as nylon yarn, and may be coated with a gas impermeable material, such as urethane, or laminated with a gas impermeable film. The extension portion 100 of the inflatable curtain 14 thus may have a substantially gas-tight construction. Those skilled in the art will appreciate that alternative materials, such as polyester yarn, and alternatives coatings, such as silicone, may also be used to construct the extension portion 100.

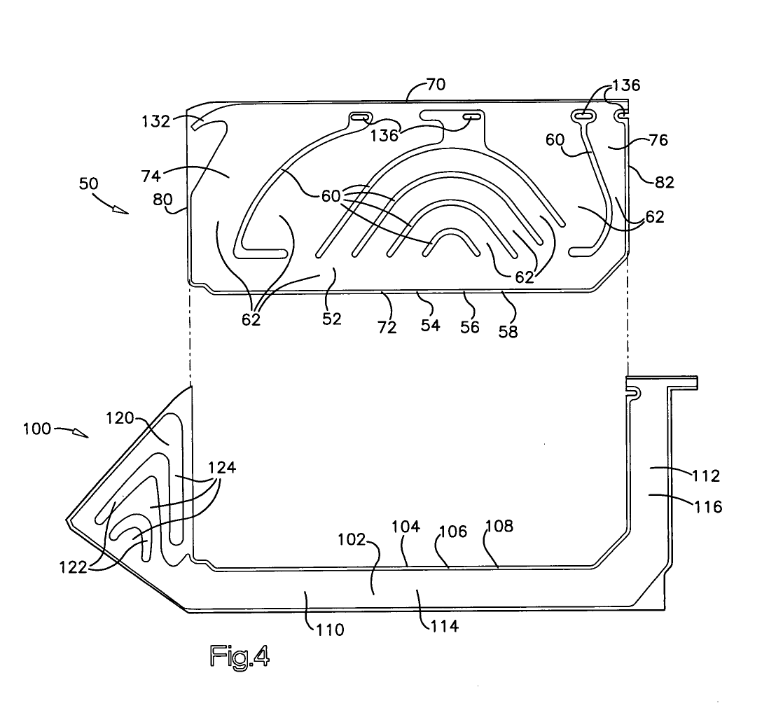

As shown in FIGS. 2 and 4, the extension portion 100 includes a lower portion 110, a rear portion 112, and a front portion 120. The lower portion 110 has a generally tubular configuration and defines an inflatable lower chamber 114 of the inflatable curtain 14. The rear portion 112 has a generally tubular configuration and defines an inflatable rear chamber 116 of the inflatable curtain 14.

The front portion 120 of the extension portion 50 has a generally triangular configuration. The front portion 120 may also include interior connections 122 in which the overlying panels 102 are interconnected within the front portion. The interior connections 122 form non-inflatable portions of the front portion 120. The interior connections 122 also help define inflatable chambers 124 of the front portion 120. The configuration of the interior connections 122, and thus the chambers 124, may vary depending on a variety of factors, such as the architecture of the vehicle 12, the position of the inflatable curtain 14 and/or front portion 120 in the vehicle, and the desired extent or coverage of the inflatable curtain and/or front portion.

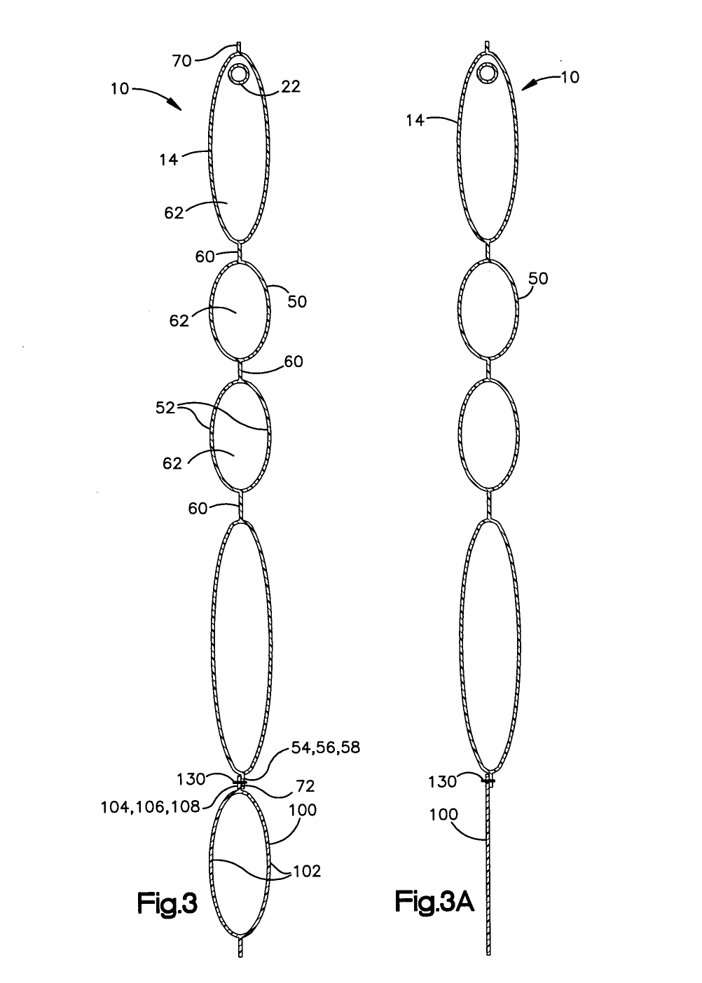

According to the present invention as illustrated in FIGS. 2-4, the main portion 50 and the extension portion 100 are separate portions that are interconnected with each other to form the inflatable curtain 14. As shown in FIGS. 2 and 3, the inflatable curtain 14 includes means 130, such as stitching, that interconnects the seam 58 of the main portion 50 with the seam 108 of the extension portion 100. Alternative means, such as ultrasonic welding, may also be used to interconnect the seams 58 and 108. Also, the seams 58 and 108 may include selvage, which may help form a portion or portions of the interconnected seams.

When the extension portion 100 is interconnected with the main portion 50, the rear portion 112 of the extension portion extends along the rear edge 82 of the main portion. The lower portion 110 of the extension portion 100 extends along the lower edge 72 of the main portion 50. The front portion 120 of the extension portion 100 extends along the front edge 80 of the main portion 50. In the configuration of FIGS. 2 and 3, the extension portion 100 thus forms the forwardmost, lowermost, and rearwardmost extent of the inflatable curtain 14.

The fill tube 22 extends through the rear portion 112 of the extension portion 100 and into the main portion 50 through the rear edge 82 of the main portion. The fill tube 22 extends along the upper edge 70 of the main portion 50 and terminates in a pocket 132 formed by the perimeter connection 56 of the main portion. The connectors 28 extend around the fill tube 22 and through respective apertures 136 in the inflatable curtain 14 and are used to connect the curtain and fill tube to the side structure 16 of the vehicle 12.

As shown in FIG. 2, one of the connectors 28 is positioned at the intersection of the upper edge 70 and rear edge 82 of the main portion 50 at the location where the seam 58 of the main portion interfaces with the seam 108 of the extension portion 100. This connector 28 extends around the fill tube 22 and around respective portions of the main portion 50 and extension portion 100. This connector 28 clamps the respective portions to the fill tube 22 and thus may help prevent inflation fluid leakage at these locations. Similarly, another connector 28 clamps the rear portion 112 of the extension portion 100 to the fill tube 22 and thereby may help prevent inflation fluid leakage at this location.

The vehicle 12 includes a sensor mechanism 150 (shown schematically in FIGS. 1 and 2) for sensing the occurrence of an event for which inflation of the inflatable curtain 14 is desired, such as a side impact to the vehicle and/or a vehicle rollover. Upon sensing the occurrence of such an event, the sensor mechanism 80 provides an electrical signal over lead wires 152 to the inflator 24. The electrical signal causes the inflator 24 to be actuated in a known manner.

The inflator 24 discharges fluid under pressure through fill tube 22, which directs the fluid into the main portion 50 and extension portion 100 of the inflatable curtain 14. The fill tube 22 may be adapted to deliver the inflation fluid at different mass flow rates into the main portion 50 and the extension portion 100 so that the portion and extension portion are pressurized at different rates. This can be achieved, for example, by altering or adjusting the size, spacing, or number of outlet apertures (not shown) in the fill tube 22. For example, the fill tube 22 may be adapted to pressurize the main portion 50 more rapidly than the extension portion 100.

The inflatable curtain 14 inflates under the pressure of the inflation fluid from the inflator 24. The inflatable curtain 14 inflates away from the roof 18 in a downward direction as shown in the drawings and in a downward direction with respect to the direction of forward travel of the vehicle 12 into the position illustrated in FIG. 2.

The inflatable curtain 14, when inflated, extends along the side structure 16 of the vehicle 12 and is positioned between the side structure and any occupant of the vehicle. The main portion 50 of the inflatable curtain 14 covers portions of the vehicle side structure that extend between the A pillar 30 and the C pillar 34 of the vehicle 12 and may overlie portions of the A pillar, C pillar, and the B pillar 32 of the vehicle. The main portion 50 may cover a portion of the vehicle side structure 16 between the front side window 40 and an occupant 270 of a front seat 154 of the vehicle 12. The main portion 50 may also cover a portion of the vehicle side structure 16 between the rear side window 42 and an occupant 276 of a rear seat 156 of the vehicle 12.

The extension portion 100 covers portions of the vehicle side structure 16 in addition to the portions covered by the main portion 50. In the embodiment of FIGS. 2 and 3, the rear portion 112 covers a portion of the side structure 16 extending adjacent and rearward of the rear side window 42 generally along the C pillar 34. The rear portion 112 extends from adjacent the vehicle roof 18 down to and below the belt line 44 of the vehicle 12. The lower portion 110 extends along the length of the inflatable curtain 14 from adjacent or near the A pillar 30 to adjacent or near the C pillar 34 and extends down to and below the belt line 44 of the vehicle 12. The front portion 120 extends from the front edge 80 of the main portion 50 to the A pillar 30 and may be connected to the A pillar. The front portion 120 extends from the roof 18 and the A pillar 30 down to and below the belt line 44 of the vehicle 12.

Those skilled in the art will appreciate that the extent and coverage of the inflatable curtain 14 in the vehicle 12 may vary. For example, the extent and coverage of the main portion 50 and the extension portion 100 may vary depending on a variety of factors, such as the architecture of the vehicle 12, the position of the inflatable curtain 14 in the vehicle, and the desired extent or coverage of the inflatable curtain.

The inflatable curtain 14, when inflated, helps to protect a vehicle occupant in the event of a vehicle rollover or a side impact to the vehicle 12. The main portion 50 and extension portion 100, combine to cover an extended area of the side structure extending from the A pillar 30 to the C pillar 34 and from the roof 18 down to below the belt line 44. The inflatable curtain 14, when inflated, helps to absorb the energy of impacts with the curtain and helps to distribute the impact energy over a large area of the curtain.

Those skilled in the art will appreciate that it may be desirable to weave the inflatable curtain 14 in a continuous weaving process in which multiple curtains are woven simultaneously on a loom. According to this practice, the inflatable curtain 14 may be woven in sheets that include rows of more than one curtain, such rows of two or three curtains. The number of curtains that may be woven in any given row is limited by the capacity of the loom and the size of the individual curtain.

For example, if the height of the entire inflatable curtain 14 of FIG. 3 is between about 500-600 millimeters and the loom is capable of weaving a sheet up to about 1800 millimeters, then the curtains may be woven in rows of three. The desired height of the inflatable curtain 14, however, may be greater than 600 millimeters, in which case, the entire curtain may be woven in rows of two.

It will thus be appreciated that, through weaving the main portion 50 separately, the main portions may be produced in rows of three, which increases the number of main portions per unit length of the sheet. The processing costs, i.e., the non-material costs associated with warping, sizing, weaving, scouring, and applying lamination or coating remain about the same regardless of whether the curtains are produced in rows of two or three. The incremental costs associated with producing the main portions 50 in rows of three as opposed to rows of two may be limited to material costs and cutting costs. As a result, weaving the main portions 50 separately may help reduce manufacturing costs of the inflatable curtain 14 on a cost per curtain basis.

Although the extension portion 100 has been illustrated thus far as being an inflatable portion of the inflatable curtain 14, it will be appreciated that the extension portion could comprise a non-inflatable piece of material (e.g., woven fabric). This embodiment is illustrated in FIG. 3A. As shown in FIG. 3A, the extension portion 100 may comprise a single sheet of material that is connected to the main portion 50 by means 130, such as stitching or ultrasonic welding. In this configuration, the lower portion 110 and rear portion 112 could comprise a strip or strips of material that are connected to the seam 58 along the lower edge 72 and rear edge 82, respectively, of the main portion 50. Also, in this configuration, the front portion 120 could comprise a generally rectangular sheet of material.

In this non-inflatable configuration, the extension portion 100 may nevertheless be connected with the fill tube 22 by the brackets 134 for the purpose of securing the extension portion to the vehicle. Of course, in this configuration, no inflation fluid would be delivered to the extension portion 100. When the inflatable curtain 14 is inflated, the extension portion 100 provides additional coverage of the side structure 16 extending down to or below the belt line 44 along the front and rear side windows 40 and 42, forward of the front edge 80 of the main portion 50, and rearward of the rear edge 82 of the main portion.

Other embodiments of the present invention are illustrated in FIGS. 5-10. The embodiments of FIGS. 5-8 are similar to the embodiment of the invention illustrated in FIGS. 1-4. Accordingly, reference numbers similar to those of FIGS. 1-4 will be utilized in FIGS. 5-8, the suffix letters “a” through “f” being associated with the reference numbers of FIGS. 5-10, respectively, to avoid confusion.

Referring to FIG. 5, the inflatable curtain 14a includes a main portion 50a and an extension portion 200. In the embodiment of FIG. 5, the extension portion 200 has a lower portion 202 that is connected to the main portion 50a along the lower edge 72a of the main portion. The extension portion 200 also has a rear portion 204 that is connected to the main portion 50a along the rear edge 82a of the main portion.

The extension portion 200 may be inflatable and thus have an inflatable tubular configuration similar to that illustrated in FIG. 3. The connection between the main portion 50a and the extension portion 200 may thus be similar or identical to the connection between the main and extension portions of FIG. 3. Alternatively, the extension portion 200 may be non-inflatable or may have non-inflatable portions and thus have a single layer configuration similar to that illustrated in FIG. 3A. The connection between the main portion 50a and any non-inflatable portions of the extension portion 200 may thus be similar or identical to the connection between the main and extension portions of FIG. 3A.

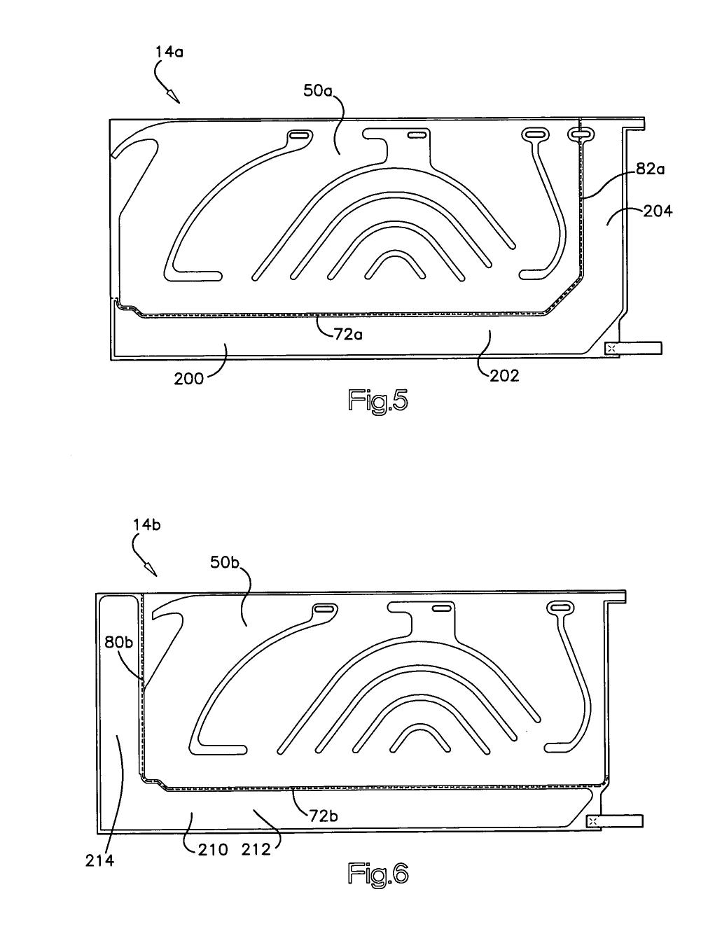

Referring to FIG. 6, the inflatable curtain 14b includes a main portion 50b and an extension portion 210. In the embodiment of FIG. 6, the extension portion 210 has a lower portion 212 that is connected to the main portion 50b along the lower edge 72b of the main portion. The extension portion 210 also has a front portion 214 that is connected to the main portion 50b along the front edge 80b of the main portion.

The extension portion 210 may be inflatable and thus have an inflatable tubular configuration similar to that illustrated in FIG. 3. The connection between the main portion 50b and the extension portion 210 may thus be similar or identical to the connection between the main and extension portions of FIG. 3. Alternatively, the extension portion 210 may be non-inflatable or may have non-inflatable portions and thus have a single layer configuration similar to that illustrated in FIG. 3A. The connection between the main portion Sob and any non-inflatable portions of the extension portion 210 may thus be similar or identical to the connection between the main and extension portions of FIG. 3A.

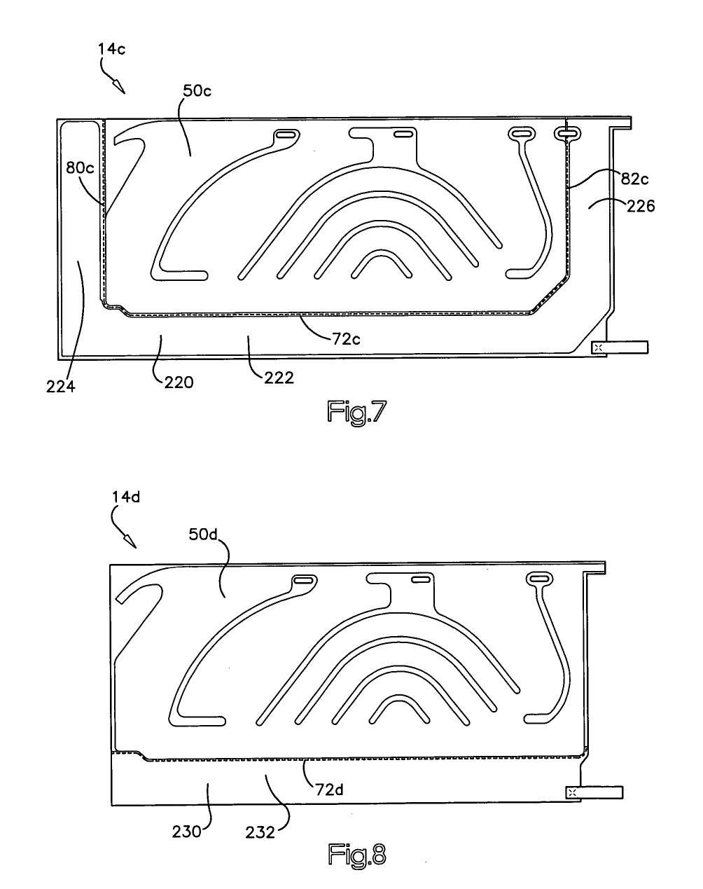

Referring to FIG. 7, the inflatable curtain 14c includes a main portion 50c and an extension portion 220. In the embodiment of FIG. 7, the extension portion 220 has a lower portion 222 that is connected to the main portion 50c along the lower edge 72c of the main portion. The extension portion 220 also has a front portion 224 that is connected to the main portion 50c along the front edge 80c of the main portion, and a rear portion 226 that is connected to the main portion along the rear edge 82c of the main portion.

The extension portion 220 may be inflatable and thus have an inflatable tubular configuration similar to that illustrated in FIG. 3. The connection between the main portion 50c and the extension portion 220 may thus be similar or identical to the connection between the main and extension portions of FIG. 3. Alternatively, the extension portion 220 may be non-inflatable or may have non-inflatable portions and thus have a single layer configuration similar to that illustrated in FIG. 3A. The connection between the main portion 50c and any non-inflatable portions of the extension portion 220 may thus be similar or identical to the connection between the main and extension portions of FIG. 3A.

Referring to FIG. 8, the inflatable curtain 14d includes a main portion 50d and an extension portion 230. In the embodiment of FIG. 8, the extension portion 230 comprises a lower portion 232 that is connected to the main portion 50d along the lower edge 72d of the main portion.

Since the extension portion 230 does not have a portion through which a fill tube may extend, the extension portion 230 may be non-inflatable or may have non-inflatable portions and thus have a single layer configuration similar to that illustrated in FIG. 3A. The connection between the main portion 50d and any non-inflatable portions of the extension portion 230 may thus be similar or identical to the connection between the main and extension portions of FIG. 3A. The extension portion 230 could, however, be inflatable if means for directing inflation fluid into the extension portion, such as a separate fill tube, are provided.

Referring to FIG. 9, the inflatable curtain 14e includes a main portion 50e and an extension portion 240. In the embodiment of FIG. 9, the extension portion 240 has a lower portion 242 that is connected to the main portion 50e along the lower edge 72e of the main portion. The extension portion 240 also has a front portion 244 that is connected to the main portion 50e along the front edge 80e of the main portion, and a rear portion 246 that is connected to the main portion along the rear edge 82e of the main portion. The extension portion 240 could have an alternative configuration in which the front portion 244 or rear portion 246 is omitted (see, e.g., FIGS. 5 and 6).

The lower portion 242 includes a lower extension 250 that extends from a lower edge 252 of the lower portion in a generally downward direction as viewed in FIG. 9. The lower extension 250 is positioned beneath the front portion 74e of the inflatable curtain 14e as viewed in FIG. 9. The lower extension 250 includes a lower edge 260, a front edge 262, and a rear edge 264 that help define the shape and extent of the lower extension. The shape and extent of the lower extension 250 could be adjusted, for example, to accommodate different vehicle configurations or to provide different coverage in the vehicle.

The extension portion 240, including the lower extension 250, is inflatable and thus may have an inflatable tubular configuration similar to that illustrated in FIG. 3. The connection between the main portion 50e and the extension portion 240 may thus be similar or identical to the connection between the main and extension portions of FIG. 3. The extension portion 240 may include non-inflatable portions and thus have portions with a single layer configuration similar to that illustrated in FIG. 3A. The connection between the main portion 50e and any non-inflatable portions of the extension portion 240 may thus be similar or identical to the connection between the main and extension portions of FIG. 3A.

The lower extension 250 of the lower portion 240 of FIG. 9 is configured to be positioned adjacent a thorax 274 of an occupant 270 of the front vehicle seat 154 (see FIGS. 1 and 2) when inflated. The lower extension 250 is dimensioned to cover an area of the side structure 16 (FIGS. 1 and 2) that extends below the belt line 44 to a position proximate an arm rest or door handle area of the side structure, generally adjacent or below the dashed line labeled 272 in FIGS. 1 and 2.

The lower extension 250 (FIG. 9) is also dimensioned to extend forwardly and rearwardly in the vehicle 12 (FIGS. 1 and 2) in order to cover an area of the side structure 16 where the thorax 274 of the occupant 270 may be positioned. The dimensions of the lower extension 250 may account for the thorax 274 of the occupant 270 being in a number of positions ranging from that associated with being seated in the forwardmost position of the front vehicle seat 154 to that associated with being seated in the rearwardmost position of the front vehicle seat. The lower extension 250, when inflated, may thus help absorb the energy of impacts with the lower extension and may help distribute the impact energy over a large area of the extension portion 240.

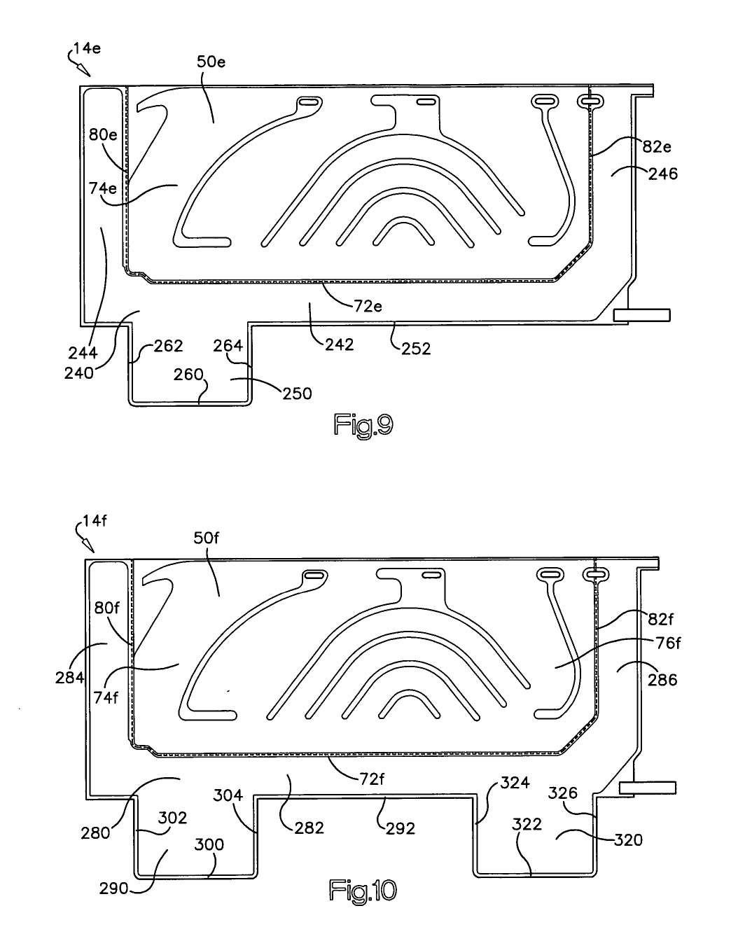

Referring to FIG. 10, the inflatable curtain 14f includes a main portion 50f and an extension portion 280. In the embodiment of FIG. 10, the extension portion 280 has a lower portion 282 that is connected to the main portion 50f along the lower edge 72f of the main portion. The extension portion 280 also has a front portion 284 that is connected to the main portion 50e along the front edge 80f of the main portion, and a rear portion 286 that is connected to the main portion along the rear edge 82f of the main portion. The extension portion 280 could have an alternative configuration in which the front portion 284 or rear portion 286 is omitted (see, e.g., FIGS. 5 and 6).

The lower portion 282 includes a front lower extension 290 that extends from a lower edge 292 of the lower portion in a generally downward direction as viewed in FIG. 10. The lower extension 290 is positioned beneath the front portion 74f of the inflatable curtain 14f as viewed in FIG. 10. The front lower extension 290 includes a lower edge 300, a front edge 302, and a rear edge 304 that help define the shape and extent of the front lower extension. The shape and extent of the front lower extension 290 could be adjusted, for example, to accommodate different vehicle configurations or to provide different coverage in the vehicle.

The lower portion 282 also includes a rear lower extension 320 that extends from the lower edge 292 of the lower portion in a generally downward direction as viewed in FIG. 10. The rear lower extension 320 is positioned beneath the rear portion 76f of the inflatable curtain 14f as viewed in FIG. 10. The rear lower extension 320 includes a lower edge 322, a front edge 324, and a rear edge 326 that together define the shape and extent of the rear lower extension. The shape and extent of the rear front lower extension 320 could be adjusted, for example, to accommodate different vehicle configurations or to provide different coverage in the vehicle.

The extension portion 280, including the front lower extension 290 and rear lower extension 320, is inflatable and thus may have an inflatable tubular configuration similar to that illustrated in FIG. 3. The connection between the main portion 50f and the extension portion 280 may thus be similar or identical to the connection between the main and extension portions of FIG. 3. The extension portion 280 may include non-inflatable portions and thus have portions with a single layer configuration similar to that illustrated in FIG. 3A. The connection between the main portion 50f and any non-inflatable portions of the extension portion 280 may thus be similar or identical to the connection between the main and extension portions of FIG. 3A.

The front lower extension 290 of the lower portion 280 of FIG. 10 is configured to be positioned adjacent the thorax 274 of an occupant 270 of the front vehicle seat 154 (see FIGS. 1 and 2) when inflated. The front lower extension 290 is dimensioned to cover an area of the side structure 16 (FIGS. 1 and 2) that extends below the belt line 44 to a position proximate the arm rest or door handle area of the side structure, generally adjacent or below the dashed line labeled 272 in FIGS. 1 and 2.

The front lower extension 290 (FIG. 10) is also dimensioned to extend forwardly and rearwardly in the vehicle 12 (FIGS. 1 and 2) in order to cover an area of the side structure 16 where the thorax 274 of the occupant 270 may be positioned. The dimensions of the front lower extension 290 may account for the thorax 274 of the occupant 270 being in a number of positions ranging from that associated with being seated in the forwardmost position of the front vehicle seat 154 to that associated with being seated in the rearwardmost position of the front vehicle seat. The front lower extension 290, when inflated, helps absorb the energy of impacts with the front lower extension and may help distribute the impact energy over a large area of the extension portion 280.

The rear lower extension 320 of the lower portion 280 is configured to be positioned adjacent the thorax 278 of an occupant 276 of the rear vehicle seat 156 (see FIGS. 1 and 2) when inflated. The rear lower extension 320 is dimensioned to cover an area of the side structure 16 (FIGS. 1 and 2) that extends below the belt line 44 to a position proximate the arm rest or door handle area of the side structure, generally adjacent or below the dashed line labeled 272 in FIGS. 1 and 2.

The rear lower extension 320 (FIG. 10) is also dimensioned to extend forwardly and rearwardly in the vehicle 12 (FIGS. 1 and 2) in order to cover an area of the side structure 16 where the thorax 278 of the occupant 276 may be positioned. The dimensions of the rear lower extension 320 may account for the thorax 278 of the occupant 276 being positioned in a number of positions ranging from that associated with being seated in the forwardmost position of the rear vehicle seat 156 to that associated with being seated in the rearwardmost position of the rear vehicle seat. The rear lower extension 320, when inflated, helps absorb the energy of impacts with the rear lower extension and may help distribute the impact energy over a large area of the extension portion 280.

It will be appreciated that the forward/rearward position of the rear vehicle seat 156 may not be adjustable. In this instance, the forward/rearward position of the rear vehicle seat 156 may not be accounted for in configuring the size and extent of the rear lower extension 320.

From the above description of the invention, those skilled in the art will perceive improvements, changes and modifications. For example, the inflatable curtain may be adapted to extend from the A pillar to the D pillar, to overlie portions of all three side windows, and to extend from adjacent the vehicle roof down to or below the vehicle belt line. Such improvements, changes and modifications within the skill of the art are intended to be covered by the appended claims.

1. An apparatus comprising:
an inflatable vehicle occupant protection device inflatable away from a roof of a vehicle to a position between a side structure of the vehicle and a vehicle occupant, said inflatable vehicle occupant protection device comprising:
an inflatable main portion inflatable to cover a portion of the vehicle side structure including a front side window of the vehicle and a rear side window of the vehicle, said main portion having upper and lower edges extending along a length of said main portion and front and rear portions spaced apart along the length of said main portion, said main portion comprising overlying panels that are interconnected to form a seam extending along said upper and lower edges and along said front and rear portions; and
an extension portion comprising a panel separate from said main portion, said extension portion being interconnected with said main portion along said seam, said extension portion covering an additional portion of the vehicle side structure.

2. The apparatus recited in claim 1, wherein said extension portion covers the additional portion of the vehicle side structure adjacent the front side window and the rear side window.

3. The apparatus recited in claim 1, wherein said extension portion comprises at least one inflatable chamber inflatable to cover the additional portion of the vehicle side structure.

4. The apparatus recited in claim 3, wherein said extension portion comprises overlying panels of material interconnected to define said inflatable chamber.

5. The apparatus recited in claim 3, further comprising:
an inflation fluid source for providing inflation fluid for inflating said main portion and said extension portion; and
a fill tube for delivering said inflation fluid from said inflation fluid source into said main portion and said extension portion, said fill tube having at least one first portion in fluid communication with inflatable chambers of said main portion, said fill tube having at least one second portion in fluid communication with said inflatable chambers of said extension portion.

6. The apparatus recited in claim 5, wherein said fill tube is adapted to deliver said inflation fluid at different mass flow rates into said main portion and said extension portion to pressurize said main portion and said extension portion at different rates.

7. The apparatus recited in claim 6, wherein said fill tube is adapted pressurize said main portion more rapidly than said extension portion.

8. The apparatus recited in claim 1, wherein said extension portion comprises an inflatable tube.

9. The apparatus recited in claim 1, wherein said extension portion extends along said lower edge and said front portion of said main portion.

10. The apparatus recited in claim 1, wherein said extension portion extends along said lower edge and said rear portion of said main portion.

11. The apparatus recited in claim 1, wherein said extension portion extends along said lower edge, said front portion, and said rear portion of said main portion.

12. The apparatus recited in claim 1, wherein said extension portion extends along said lower edge of said main portion and includes a lower extension that extends from a lower edge of said extension portion.

13. The apparatus recited in claim 12, wherein said lower extension is inflatable.

14. The apparatus recited in claim 13, wherein said lower extension when inflated extends generally downward from said lower edge of said extension portion to a position below a belt line of the vehicle proximate a portion of the side structure including at least one of an arm rest and a door handle of the vehicle.

15. The apparatus recited in claim 13, wherein said lower extension when inflated covers a portion of the side structure where a thorax of a vehicle occupant may be positioned.

16. The apparatus recited in claim 13, wherein said lower extension when inflated covers a portion of the side structure that extends from where a thorax of a vehicle occupant may be positioned in a rearwardmost position of a vehicle seat to where the thorax of the vehicle occupant may be positioned in a forwardmost position of the vehicle seat.

17. The apparatus recited in claim 12, wherein said lower extension is adapted to be positioned adjacent an occupant of a front vehicle seat of the vehicle.

18. The apparatus recited in claim 12, wherein said lower extension is adapted to be positioned adjacent an occupant of a rear vehicle seat of the vehicle.

19. The apparatus recited in claim 12, wherein said extension portion comprises a front lower extension adapted to be positioned adjacent an occupant of a front vehicle seat of the vehicle and a rear lower extension adapted to be positioned adjacent an occupant of a rear vehicle seat of the vehicle.

20. The apparatus recited in claim 1, wherein said extension portion comprises a front extension portion connected to said front portion of said main portion, said front extension portion extending from said front portion to adjacent an A pillar of the vehicle.

21. The apparatus recited in claim 20, wherein said front extension portion is connected to the A pillar of the vehicle.

22. The apparatus recited in claim 21, wherein said front extension portion comprises a plurality of inflatable chambers.

23. The apparatus recited in claim 1, wherein said main portion of said inflatable curtain comprises a plurality of inflatable chambers, at least one of said chambers having a curved configuration with a portion adapted to be positioned adjacent the front side window and a portion adapted to be positioned adjacent the rear side window when said inflatable curtain is inflated in the vehicle.

24. The apparatus recited in claim 1, wherein said additional portion covered by said extension portion comprises a portion of the side structure extending below a belt line of the vehicle adjacent both the front and rear side windows of the vehicle.

25. The apparatus recited in claim 1, wherein said seam comprises selvage extending along said edges of said main portion, said extension portion being interconnected with said selvage.

26. The apparatus recited in claim 1, wherein said extension portion is formed as a single non-inflatable panel of material.

27. The apparatus recited in claim 1, wherein said extension portion is sewn to said main portion along said seam.

28. An inflatable vehicle occupant protection device inflatable away from a roof of a vehicle to a position between a side structure of the vehicle and a vehicle occupant, said inflatable vehicle occupant protection device comprising:
an inflatable main portion inflatable to cover a portion of the vehicle side structure including a front side window of the vehicle and a rear side window of the vehicle, said main portion having upper and lower edges extending along its length and front and rear edges extending between said upper and lower edges, said main portion being woven as a single piece of material and comprising a woven seam extending along said edges; and
an extension portion woven as a single piece of material separate from said main portion, said extension portion being interconnected with said main portion along said seam, said extension portion covering an additional portion of the vehicle side structure.

29. The inflatable vehicle occupant protection device recited in claim 28, wherein said extension portion comprises an inflatable tubular chamber.

30. The inflatable vehicle occupant protection device recited in claim 28, wherein said extension portion is formed as a single non-inflatable panel of material.

31. An apparatus for helping to protect an occupant of a vehicle that has a side structure including front and rear side windows and a roof, said apparatus comprising:
an inflatable curtain that is inflatable away from the vehicle roof to a position adjacent the side structure of the vehicle, said inflatable curtain comprising:
an inflatable main portion inflatable to cover a portion of the vehicle side structure between the front side window and a front vehicle occupant and between the rear side window and a rear vehicle occupant, said main portion having upper and lower edges extending along its length and front and rear edges extending between said upper and lower edges, said main portion comprising overlying panels that are interconnected to form a seam extending along said edges; and
an extension portion comprising a separate panel of material interconnected with said main portion along said seam and extending along at least one of said edges, said extension portion covering an additional portion of the vehicle side structure.

32. The inflatable vehicle occupant protection device recited in claim 31, wherein said extension portion comprises an inflatable tubular chamber.

33. The inflatable vehicle occupant protection device recited in claim 31, wherein said extension portion is formed as a single non-inflatable panel of material.

34. An apparatus comprising:
an inflatable vehicle occupant protection device inflatable away from a roof of a vehicle to a position between a side structure of the vehicle and a vehicle occupant, said inflatable vehicle occupant protection device including a lower edge extending along a length of said inflatable vehicle occupant protection device; and
a lower extension portion extending from said lower edge along a portion of the length of said inflatable vehicle occupant protection device, said lower extension portion being adapted to cover a portion of the side structure where a thorax of a vehicle occupant may be positioned.

35. The apparatus recited in claim 34, wherein said lower extension when inflated covers a portion of the side structure that extends from where a thorax of a vehicle occupant may be positioned in a rearwardmost position of a vehicle seat to where the thorax of the vehicle occupant may be positioned in a forwardmost position of the vehicle seat.

36. The apparatus recited in claim 34, wherein said lower extension portion when inflated extends generally downward from said lower edge to a position below a belt line of the vehicle proximate a portion of the side structure including at least one of an arm rest and a door handle of the vehicle.
